(19) United States
(12) Patent Application Publication
(10) Pub. No.: US 2007/0243238 A1
(43) Pub. Date: Oct. 18, 2007

(54) Treating mouth sores with patches adhered to teeth

(76) Inventor: Jeffrey T. Haley, Mercer Island, WA (US)

Correspondence Address: GRAYBEAL, JACKSON, HALEY LLP, 155 - 108TH AVENUE NE, SUITE 350, BELLEVUE, WA, 98004-5973, US

(21) Appl. No.: 11/639,982

(22) Filed: Dec. 15, 2006

Related U.S. Application Data
(60) Provisional application No. 60/792,121, filed on Apr. 13, 2006.

(51) Int. Cl. A61K 36/48; A61K 31/58; A61K 31/573; A61K 31/704

(52) U.S. Cl. 424/448; 424/757; 514/33; 514/171; 514/176

(57) ABSTRACT

A method for treating a mouth sore opposite a tooth by adhering an oral patch on the tooth to speed healing and relieve pain. The oral patch is placed either directly on the portion of the tooth that comes into contact with the sore or is first place onto the sore and then adhered to the tooth. The patch may be a blob of hydrophilic gums. If certain medications are applied to a mouth sore using an oral patch that delivers the medication for at least 30 minutes and the patches are used for at least two or more hours per day, the method reduces the healing time for mouth sores from typical 10-14 days to 1-5 days. The method can be used with various antimicrobials, glucocorticoids or anthihistamines incorporated into the patch that reduce inflammation or speed the healing of mouth sores.

### RELATED APPLICATIONS

The present application claims priority from PCT/02/35399 (agent ref 0795-010-04) application filed Nov. 5, 2002, via co-pending Ser. No. 10/772,099 (agent ref 0795-010-03) which claimed priority from it and was filed Feb. 4, 2004, and from U.S. provisional patent applications No. 60/792,121 (agent ref 0795-010-22) filed Apr. 13, 2006.

### BACKGROUND

People often bite their lip or cheek, causing a mouth sore at the point of tooth contact. Also, their teeth sometimes get chipped or otherwise develop abrasive edges. These abrasive edges can rub against surrounding tissues on the tongue, lips, or cheek resulting in a sore opposite the tooth. Sometimes mouth sores form opposite a tooth for reasons unrelated to the tooth. There is a need for methods and products that can be used in promoting healing or alleviating pain associated with sores that are opposite teeth.

### SUMMARY

The present invention fills this need by providing for oral patches and methods of applying oral patches to teeth to alleviate pain and promote healing of mouth sores that are opposite the teeth. In particular, a method is provided for treating a sore in the mouth by placing an oral patch at a location on a tooth that contacts a sore such that the patch becomes adhered to the tooth, and allowing the patch to remain adhered to the tooth for more than 30 minutes. In an alternative embodiment, a patch is placed first on the sore at a location that comes into contact with the tooth and then held in contact with the sore and the tooth until the patch adheres to the tooth.

A preferred oral patch is made of hydrophilic gums which may be formed as a blob of hydrophilic gums.

### BRIEF DESCRIPTION OF THE DRAWINGS

FIG. 1a shows a side view of an oral patch that completely dissolves.

FIG. 1b shows a top view of the same oral patch.

FIG. 2 shows an oral patch adhering onto a tooth preventing the tooth from coming into contact with a sore on the cheek of a patient.

### DETAILED DESCRIPTION

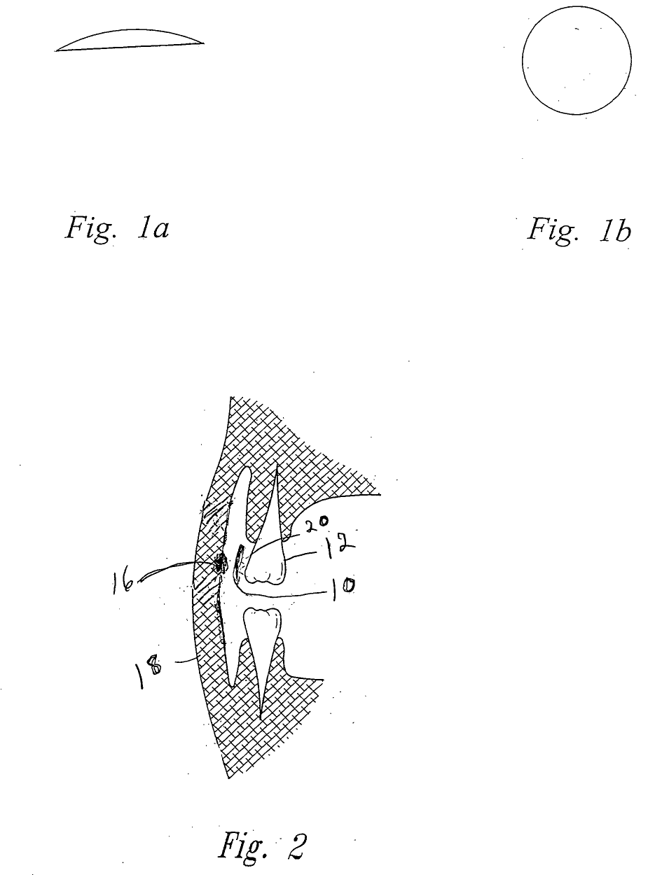

FIG. 1 shows an oral patch that completely dissolves. It has a feel and texture like gummy candies. It is made with slowly dissolving hydrocolloids so that that it typically lasts in the mouth for at least one to six hours. The patch can be formed in the shape of a tablet or a lozenge or a wafer or any other desired shape. The preferred shape is a lentil as shown in FIG. 1a.

FIG. 2 shows an oral patch 10 attached to a tooth 12. Patch 10 is attached to tooth 12 to prevent contact of the tooth 12 with sore 16 on the inner surface of cheek 18. Sore 16 may have been caused or irritated by tooth 12 having a jagged spot or chipped edge on the cheek side 20 of tooth 12. Users apply the patch to the tooth opposite the mouth sore so that the patch is touching the mouth sore most of the time and is stuck to the tooth. As it softens, the patch settles onto the tooth. It will completely dissolve in 1-9 hours. All this time it supplies medication to the sore.

A preferred oral patch to treat a sore opposite a tooth is made of two primary components.

The first component is a porous molecular network formed as a unitary solid structure that remains a solid at human mouth temperatures. In preferred embodiments, this network is elasto-plastic (elastic or plastic or a combination thereof), giving it a soft feel in the mouth, in contrast to being crumbly or a paste. The network is preferably hydrophilic so that, even when applied to a wet mucosal surface in the mouth, it will tend to adhere by absorbing moisture from the mucosal surface. Preferably, the network slowly dissolves in saliva so that the patch or blob merely dissipates over time and the patch or blob never has to be removed from the mouth.

The second component is a hydrophilic polymer that is liquid at human mouth temperatures which is distributed throughout the pores of the network. Because the polymer is hydrophilic and liquid at human mouth temperatures, it will adhere very well to wet surfaces inside the mouth and is quite soft which provides a soothing feeling to any sensitive tissue such as sores. Also because the patch is soft when it is wet, the patch will adhere to a tooth.

Instead of placing the patch directly on the tooth, the patch 20 may be placed onto sore 16. The adherent oral patch will adhere to the cheek or lips without the user first drying saliva from the tissue. If the patient merely places the oral patch in his or her mouth and holds it in the desired location for 10 to 40 seconds, it will then adhere to the tissues that it has been touching without movement, even though those tissues are wet. This is far easier for patients to use than requiring that the tissue first be dried with a towel before the adherent oral patch is placed. If the patch is held in contact with both the sore and the tooth it will also become adhered to the tooth. It may then be pulled free of the sore, remaining adhered to the tooth.

A desired medication may also located within the pores of the network along with the hydrophilic adherent polymer.

The network component may be comprised of a thermo gel having a melting temperature higher than human mouth temperatures. Preferably, the thermo gel is elasto-plastic, such as formed by a mixture of the hydrogels konjac gum and xanthan gum dissolved in hot water and then cooled to form an elasto-plastic gel. Alternatively, the network may be comprised of a complex carbohydrate, such as cellulose, pectin, maltodextrin, or starch from potato, rice, corn, or wheat. Also, the network may be comprised of a hydrogel with a melting temperature higher than temperatures in the human mouth formed of amino acids, such as peptides.

In preferred embodiments, the hydrophilic adherent polymer gels to a solid at room temperatures. This allows the oral patch to be removed from the mouth and placed on a smooth surface, such as a plastic bag. Because the hydrophilic polymer then gels, the oral patch again becomes handleable with the fingers to return it to the mouth without being too sticky to handle or leaving a residue on the fingers or on the plastic bag. In one such embodiment, the hydrophilic polymer is protein gelatin rendered from animal tissue, which solidifies at just below mouth temperatures and remains a solid even at clothes pocket temperatures so it will not melt in a pocket.

In a method for producing an oral patch, ingredients for forming the porous network, molecules of the hydrophilic adherent polymer, molecules of the medication, and water are mixed together. The mixture is heated to dissolve all ingredients, either before the ingredients are added together or after they are added together, and the mixture is then cooled, thereby causing the ingredients for forming a network to form the porous network as a unitary solid structure having the medication and the hydrophilic adherent polymer within its pores. Before it is cooled, the hot mixture may be deposited into a mold of a suitable shape to form the preferred unitary solid structure. The mold may be formed in powdered starch, as is well known in the candy making industry for forming gumdrops. Alternatively, the mold may be formed of a rigid material such as metal or plastic. If the mold is thin plastic or aluminum, it may also serve as packaging for delivery of the oral patch to the consumer.

Many different compositions can be used to form the network. For ease of manufacturing, it is convenient if the network is comprised of a thermo gel having a melting temperature higher than human mouth temperatures. This allows the entire mixture to be a liquid at temperatures far above human mouth temperatures and allows the network to be formed by cooling the mixture such that the thermo gel forms the desired network by a gelation process. The temperature at which the gel forms can be lower than human mouth temperatures, provided the temperature at which it melts again is higher than human mouth temperatures.

Readily available materials that form such a gel include agar, in various forms, carrageenan, in most of its forms particularly kappa carrageenan, konjac gum, locust bean gum, and xanthan gum. All of these materials form a thermo gel that is sufficiently elastic or plastic or a combination thereof for the network to feel soft in the human mouth if it is adequately hydrated. If water is dried out of the network, it will become hard and will produce an unattractive feel when placed in contact with sensitive tissues, such as mouth sores. To prevent the network from drying out, it may be packaged with a hermetic seal or a non-evaporating plasticizer, such as glycerol (glycerin) may be added. However, the more glycerol is added the less adherent the oral patch will be.

Synthetic hydrogels may be used for either the network that does not melt at mouth temperatures or the adherent polymer. Protein-based hydrogels are usually prepared using proteins extracted from natural sources, but they may be synthesized, such as with diblock copolypeptide amphiphiles, as taught by Nowak, et. al, “Rapidly Recovering Hydrogel Scaffolds From Self-Assembling Diblock Copolypeptide Amphiphiles”. Nowak, A. P.; Breedveld, V.; Pakstis, L.; Ozbas, B.; Pine, D. J.; Pochan, D.; Deming, T. J. Nature, 2002, 417, 424-428. The use of synthetic materials allows adjustment of copolymer chain length and composition. Synthetic hydrogels may also be made from polysaccharides and synthetic block copolymers which form thermoreversible gels and allow the solubilisation of hydrophobic medications for controlled release, as taught by Williams, Pa., at the Centre for Water Soluble Polymers, North East Wales Institute, Plas Coch, Mold Road, Wrexham, Wales.

Instead of forming the network with a true hydrogel, the network may be formed with a complex carbohydrate, such as cellulose, pectin, starch, maltodextrin or other polysaccharides. Forming of hydrated network structures out of such materials is well known in the candy making industry for making gummy candies. Or the network may be formed with a combination of a true hydrogel and a complex carbohydrate.

For desirable adhesion and erosion characteristics in an adherent oral patch, the mixture may include a hydrophilic adherent polymer that is liquid at human mouth temperatures. Collagen molecules, such as gelatin rendered from animal protein, such as from pork or cattle skin or from fish, serve very well as this ingredient. Collagen molecules tend to adhere very well to the tissues of the mouth lining, which themselves, are collagen molecules. The collagen molecules may be partially hydrolyzed, making them shorter and lower in molecular weight, in the form of commercially available gelatin.

A suitable hydrophilic polymer is gelatin rendered from animal protein, such as from pork skin or cattle hooves or from fish. These gelatins are graded according to “bloom strength” which refers to the strength of the gel that is formed. Gelatin having a higher bloom strength is preferred for the adherent oral patch because it also has a higher viscosity in liquid form. The high viscosity in liquid form prevents the gelatin molecules from escaping the network substantially faster than the network erodes, and the high viscosity better retains the medication for slow release. The highest commercially available bloom strength, 250, is preferred.

The adherent oral patch is suitable for use with all of the medications mentioned in U.S. Pat. No. 6,139,861 issued to Friedman, including steroids, such as a glucocorticoid steroids, and non-steroidal anti-inflammatory drugs such as naproxen sodium, ibuprofen, acetaminophen, and ketoprofen. The medication may also be an antimicobial, such as an anti-fungal for treatment of candida organisms (thrush), such as nystatin, clotrimazole, miconazole, or fluconazole. Such medications also include medications such as tetracycline, penicillin, amoxicillin, triclosan, licorice root extract or amlexanox.

If the network is formed of a hydrogel as described above, the oral patches may be manufactured by processes well known in the candy making industry. The process is to form a well-hydrated mixture at temperatures just below the boiling temperature of water so that water does not boil off and yet the hydrogels are fully activated for gelling when the product is cooled. In this process, the network can be formed of a combination of a true hydrogel such as xanthan gum with locust bean gum or with konjac gum and a complex carbohydrate such as cellulose or pectin or starch. For the medication licorice root extract, an effective ratio by weight is 56% water, 16% gelatin, 11% licorice extract, 10% cellulose, 4.8% glycerol, and 2.2% gums such as kappa carrageenan or xanthan gum plus locust bean gum or konjac gum heated to 200 degrees F.

For most applications, most users prefer that the oral patches be medium dry to dry. With this starting dryness, the oral patches are more adherent and have more integrity so they can be removed for talking or eating and then replaced. The only drawback to this dryness is that the oral patch becomes hard when it dries, giving the oral patch a less soothing feel. It is also less conforming and therefore does not stick well to hard surfaces such as gums and teeth. When the oral patch is placed in contact with delicate tissue, such as a large sore, most users prefer that the oral patch be moist and soft. Thus, it is preferable to package the oral patches with a film that allows moisture to pass so moisture can easily be added to or removed from the oral patches without removing them from the packaging. If the packaging film is a barrier to germs, this allows the oral patches to remain sterile and not grow mold even when they are moist. Effective films are cellophane, polystyrene, poybutadiene, polyamide, Tyvek (matted polyethylene threads) and expanded films such as Goretex. Polyamide with a thickness of 0.7 mil to 1.0 mil is effective. Allowing such a package to sit for a day or two with a few drops of water on the package is sufficient to hydrate the oral patch inside. Conversely, allowing the package to sit on a shelf in a dry room for one to three days is sufficient to dry out the oral patch.

To cause the patch to dissolve very slowly in saliva, a binder that dissolves slowly in saliva is incorporated. Binders that have been tested and found to work well include carrageenan (preferably kappa), xanthan gum combined with konjac gum, and agar. Another useable gum is gum arabic. Other gums similar to those listed, such as locust bean gum which has properties similar to konjac gum, and guar gum should also work.

In addition to causing the patch to dissolve very slowly in the mouth, the binder also moderates any strong flavors by spreading out over a long period of time the release of that flavor. Consequently, sweeteners and other products to mask strong flavors are not required, although some users prefer a small amount of sweetener and some also prefer the addition of anise or other flavors.

Instead of depositing a hydrated mixture onto cornstarch molds, the mixture may be deposited as an array of hot, viscous drops or blobs onto a sheet of high temperature plastic or coated paper. The drops are allowed to cool and then the sheets of plastic or coated paper with the drops on them are dried in a drying room or oven till the water activity level less than 0.8. The product is sold still adhered to the plastic or paper and the user pulls it off the plastic or paper.

A preferred process for producing oral patches suitable for binding to a tooth begins by mixing ingredients including water and hydrophilic polymers, such as food gums or synthetic polymers such as carboxymethylcellulose, hydroxymethylcellulose, polyacrylic acid, or carbopol-934. Other ingredients that should be released as the finished product dissolves or erodes, such as flavoring, coloring, a medication, or and ingredient to aid erosion/dissolution, may be added. To retain flexibility in the finished product, a non-evaporating plasticizer, such as glycerol (glycerin) may be added within the structural network formed by the polymers. However, the more glycerol is added the less adherent the oral patch will be.

For ease of manufacturing, it is convenient if the polymers form a thermo gel having a melting temperature higher than human mouth temperatures. This allows the entire mixture to be a liquid at temperatures far above human mouth temperatures and allows the deposited blob to become strong by cooling the mixture such that the thermo gel forms a strong blob by a gelation process. The temperature at which the gel forms can be lower than human mouth temperatures, provided the temperature at which it melts again is higher than human mouth temperatures.

Readily available materials that form such a gel include many of the food gums, such as agar, in various forms, carrageenan, in most of its forms, particularly kappa carrageenan, konjac gum, locust bean gum, and xanthan gum. All of these materials form a thermo gel that is sufficiently elastic or plastic or a combination thereof to feel soft in the human mouth if it is adequately hydrated. However, gelation after deposition is not required. The blobs can remain tacky and viscous while they are drying.

As or after the mixture is prepared, it may be heated to reduce viscosity or to activate or prevent gelation or to minimize growth of bacteria during manufacturing. For the latter purpose, a temperature at or above 140 degrees F. is preferred. To avoid complications of boiling, the mixture should be kept below the boiling temperature, which because molecules are dissolved in the water, may be about 215 degrees F. Within this range, the temperature may be selected by manufacturing considerations. A temperature range of 160 degrees to 180 degrees has been found effective.

The hot mixture is poured or squirted into molds. The molds be may open top molds or closed molds. Open top molds may be formed by pressing a plug into powdered starch such as cornstarch or may be formed in a tray for packaging the products such as thermo formed PVC or PET or a cold press laminate of aluminum and PVC with a thin layer of polyamide for strength. Closed molds may be used such as in an injection-molding machine. Because the mixture typically requires about 2 hours to form a strong enough oral patch for de-molding, it is preferable to intermittently move trays of two-part molds, upper and lower, under pump depositor injector nozzles. The nozzles fit into holes in the upper mold located at the center of each oral patch. After de-molding, the upper molds are used again for another batch. The lower molds may be plastic lined, in which case the plastic becomes a part of the final packaging. A suitable size for each oral patch is 0.8 grams poured into the mold.

The height of each blob relative to its width is a function of the viscosity of the mixture as it is deposited. The width and height of each blob are adjusted by changing viscosity and the amount of material in each blob to achieve a preferred shape. If the blobs are deposited in powdered starch, the starch absorbs some of the excess water and the blobs are further dried in a drying room before being removed from the starch, packaged in a hermetic seal, and sterilized with gamma radiation or heat and pressure in a retort.

If the oral patches are deposited in molds formed in a tray, the tray is stored in a drying room until the oral patches lose a suitable amount of moisture. A suitable method of drying in trays is to expose them without convection to room temperature and humidity for 3 days or, with convection, for 24-72 hours. In the drying process, the oral patches lose about 47% of their weight, so an oral patch that started at 0.8 grams poured into the mold becomes 0.42 grams. The trays are then sealed with a film or foil lid that is adhered by conventional heat-sealing techniques and the entire package is sterilized with gamma radiation or heat and pressure in a retort.

Depositors used in the candy making industry are suitable, such as depositors made by NID of Australia. One process is to form a well-hydrated mixture at temperatures above the activation temperature and below the boiling temperature of water so that water does not boil off and yet the hydrogels are fully activated for gelling when the product is cooled. In this process, the mixture can be formed of a combination of a true hydrogel such as xanthan gum with locust bean gum or with konjac gum and a complex carbohydrate such as cellulose or pectin or starch. For a patch with the medication licorice root extract, an effective ratio by units of weight is 9690 units water, 1640 units gelatin, 724 units licorice extract, 960 units cellulose, and 274 units of xanthan gum mixed with konjac gum heated to between 140 and 200 degrees F.

For cost reasons, it is advantageous to deposit the blobs directly onto a thermoplastic sheet that can be used a part of the final package. Suitable materials include, nylon, polystyrene, polyethylene, and polypropylene. A suitable thickness is 0.030 inches. The sheets can then be placed in a drying room and then covered with a sheet of lid stock such as metal foil or thin plastic coated with sealant. The process can be done on individual sheets on individual trays of any suitable size for running through the depositor or can be done with continuous roll sheet material which runs through a long drying chamber.

If the oral patches are deposited onto a tray (or sheet on a tray), the tray is stored in a drying room until the oral patches lose a suitable amount of moisture. A suitable method of drying is to expose the blobs to room temperature and humidity for 3 days with or without convection. If convection is used, the sheets can be closely stacked. Refrigeration type dehumidifiers can lower the humidity to 30% which is quite sufficient. The drying process can be taken to any desired level of dryness, such as dry enough that microorganisms will not grow.

The drying process converts a blob to a thin disc. For an oral patch, the shape shown in FIG. 1(b) is preferred. The thickest point is in the center and it gently tapers toward the edges until the final edge is rounded, not square or sharp. The ratio of maximum thickness to diameter can be adjusted by changing the viscosity of the mixture.

To prevent the discs from gaining or losing moisture, they may be packaged with a hermetic seal. Alternatively, the oral patches may be packaged with a base or lidstock film that allows moisture to pass so moisture can easily be added to or removed from the oral patches without removing them from the packaging. If the packaging film is a barrier to germs, this allows the oral patches to remain sterile and not grow mold even when they are moist. Effective films are cellophane, polystyrene, polybutadiene, polyamide, Tyvek (matted polyethylene threads) and expanded films such as Goretex. Polyamide with a thickness of 0.7 mil to 1.0 mil is effective. Allowing such a package to sit for a day or two with a few drops of water on the package is sufficient to hydrate the oral patch inside. Conversely, allowing the package to sit on a shelf in a dry room for one to three days is sufficient to dry out the oral patch.

The base sheets are sealed with a film or foil lid that is adhered by conventional sealing techniques. Hot or cold sealant may be used by printing the sealant in a suitable pattern onto the base sheet or onto the lid stock. Or the lid stock can be coated with heat sealant and a platen with recesses can press the sheets together onto an anvil where the anvil or the platen is heated. The recesses prevent the dried discs from being squashed or excessively heated. The platen and anvil may be in the form of rollers with either or both of them heated, or one of them may be flat while the other is flat except for recesses. The platen can be pressed with a hydraulic press or a pneumatic press.

If the lid stock is aluminum foil, that discs may be released from the packaging by pushing them through the foil. Pressing flat lid stock onto a flat sheet with discs between them requires that the base sheet or the top sheet (or both) flexes (stretches) to avoid wrinkles. The aluminum will be most stretchy if it is not tempered. Although standard aluminum lid stock coated with heat sealant is high temper, it is preferable to order custom foil with zero temper. The sheets that have been sealed together are cut into cards of suitable shape, such as by die cutting. Cards that easily fit in a pocket are preferred, such as 2.3 inches by 3.4 inches. The entire package may be sterilized with gamma radiation or heat and pressure in a retort.

For medication to be placed in the patch, any of the above-mentioned antimicrobials, or similar antimicrobials, may be used. Using an extract of licorice root with glycyrrhetinic acid presents significant advantages over pharmaceutical antibiotics because, in the quantities that a person could use per day, no side affects have been discovered. The preferred quantity of glycyrrhetinic acid in each patch that lasts 1 to 6 hours is 5 to 10%.

For treatment of the tongue, most users stick a patch that releases medication on both sides to the closest tooth. This works particularly well at night.

Users with abrasive teeth apply the patch to the tooth opposite the canker sore so that the patch is touching the canker sore most of the time and is stuck to the teeth. As it softens, the patch settles onto the tooth. It will completely dissolve in 1-9 hours. All this time it supplies medication to the sore.

To alleviate inflammation in the sores topical glucocorticoids or antihistamines can be incorporated into the patch to treat the resultant contact dermatitis. Examples of suitable topical glucocorticoids include but are not limited to those described on pages 1799 and 1800 of Goodman & Gilman's “The Pharmacological Basis of Therapeutics” 10^{th} Edition (McGraw-Hill, 2001). Representative examples of the topical glucocorticoids that can be incorporated into oral patches to treat mouth sores caused by teeth include dexoximethasone, triamcinolone acetonide, mometasone furoate, and hydrocortisone.

Antihistamines can also be incorporated in the patch. Examples of antihistamines (H₁-Receptor Antagonists) that can be incorporated into the patch to alleviate the contact dermatitis caused by the tooths include but are not limited to doxepin hydrochloride, diphenhydramine hydrochloride, pyrilamine maleate, azelastine hydrochloride, and levocabastine hydrochloride.

If the user catches the sore early, shorter treatment is required. The sore will often start in a small cut. Some users report that if they apply one patch to a cut for 1-4 hours before there is any sensation of a sore, then they will not get a mouth sore from the cut. Other times, the sore starts with a feeling that the mucous layer is becoming too thin in a spot before it becomes painful. Some users report that if they apply one patch to that spot, no mouth sore develops. Some users report that if they begin applying the patch when the mouth sore is very small and barely painful, only 24 hours of treatment is required, but if they wait until the sore is as large as a tomato seed, then they need 48 hours of treatment before it starts to heal.

Because use of antibiotics, antibacterials, and other pharmaceutical antimicrobials causes adverse side effects, experimentation with these medications has been limited. However, sufficient experimentation was done with amoxicillin on conventional patches to determine that it works quite well. Conventional amoxicillin powder for preparation of oral suspension was used, which includes sugars and other excipients. The sugars act as a binder to the oral patch. The oral suspension is massaged into the patch and dried. Preferably, the amoxicillin powder is mixed into a stronger than normal liquid before being added to the patch.

The following show non-limiting examples of oral patches used for the above-described method for treating sores opposite a tooth.

### EXAMPLE 1

A preferred patch for use in the method of the present invention is comprised of the following ingredients.

| | |
|---|---|
| Gelatin | 50% |
| Glycyrrhetinic acid (potassium salt) | 7% |
| Benzocaine | 8% |
| Konjac-xanthan gum | 8% |
| Glycerol | 5% |
| Short cellulose fibers | 15% |
| Calcium H phosphate | 5% |
| Xylitol | 4% |

### The CaHPO4 is to neutralize nearby acids and release ions that are good for teeth.

### EXAMPLE 2

The following shows an example for ranges of ingredients in one example of a patch and an example of how a patch can be produced.

Active for numbing: benzocaine—a light amount, 3% to 8%, 5% is preferred.
Other active: Glycyrrhetinic acid, 5% to 10%, 7% preferred, or triclosan, 0.3% to 3%, 0.5% is preferred.
Structural ingredients:
30-60% gelatin, 55% preferred
5%-8% konjac gum and xanthan gum premixed from Gum Technologies, 7% preferred
8%-20% glycerol (glycerin), 15% preferred
0%-30% cellulose fiber, 15% preferred
The percentages are of dry ingredients. Add water to preferred consistency, 50-80% of the mixture, 70% is preferred. Heat to above 140 degrees F., stir, for more than 30 minutes for full hydration of gums. Deposit with a 1-3 mm nozzle onto polypropylene plastic sheet blobs that are 8-13 mm across, preferably 10 mm. Dry with convection until equilibrates with air of 45% humidity or less (preferred 40% humidity) at room temperature.

While particular embodiments of the invention have been described above the scope of the invention should not be limited by the above descriptions but rather limited only by the following claims.

1. A method for treating a mouth sore in a human, comprising:
placing an oral patch at a location on a tooth that contacts the sore such that the patch becomes adhered to the tooth; and
allowing the patch to remain adhered to the tooth for more than 30 minutes.

2. The method of claim 1 wherein the patch stays on the tooth for at least two or more hours.

3. The method of claim 1 wherein the patch dissolves in saliva over more than 30 minutes.

4. The method of claim 1 wherein the patch releases an anti-inflammatory medication.

5. The method of claim 4 wherein the anti-inflammatory medication is an extract of licorice root.

6. The method of claim 5 wherein the extract of licorice root comprises glycyrrhetinic acid.

7. The method of claim 6 wherein the glycyrrhetinic acid is incorporated into the patch in the form of a water soluble salt of glycyrrhetinic acid.

8. The method of claim 4 wherein the anti-inflammatory medication is a glucocorticoid.

9. The method of claim 8 wherein the glucocorticoid is selected from the group consisting of dexoximethasone, triamcinolone acetonide, mometasone furoate, and hydrocortisone or salt thereof.

10. The method of claim 1 wherein the patch releases an anesthetic medication.

11. The method of claim 10 wherein the anesthetic medication is benzocaine.

12. The method of claim 1 wherein the patch releases triclosan.

13. The method of claim 1 wherein the patch is comprised of hydrophilic gums that dissolve in saliva.

14. The method of claim 13 wherein the hydrophilic gums comprise at least one of: gelatin, gelatinized starch, alginate gum, xanthan gum, konjac gum, guar gum, gellan gum, locust bean gum, pectin, polyvinylpyrollidone, polyvinylacetate, hydroxy-propyl-cellulose, hydroxy-methyl-cellulose, hydroxy-ethyl-cellulose, carbomer, and carbopol.

15. The method of claim 1 wherein the patch does not contain any structural ingredients that do not erode or dissolve in saliva.

16. The method of claim 1 further comprising:
first placing the patch on the sore at a location that comes into contact with the tooth and allowing the patch to contact the tooth and the sore simultaneously until the patch adheres to the tooth.

17. A method for providing a treatment for a mouth sore in a human, comprising:
providing an oral patch with instructions to place the patch at a location on a tooth that contacts the sore such that the patch becomes adhered to the tooth and allow the patch to remain adhered to the tooth for more than 30 minutes.

18. The method of claim 17 wherein the instructions further instruct:
first place the patch on the sore at a location that comes into contact with the tooth and then allow the patch to contact the tooth and the sore simultaneously until the patch adheres to the tooth.

19. The method of claim 17 wherein the patch is comprised of hydrophilic gums that dissolve in saliva.

20. The method of claim 19 wherein the hydrophilic gums comprise at least one of: gelatin, gelatinized starch, alginate gum, xanthan gum, konjac gum, guar gum, gellan gum, locust bean gum, pectin, polyvinylpyrollidone, polyvinylacetate, hydroxy-propyl-cellulose, hydroxy-methyl-cellulose, hydroxy-ethyl-cellulose, carbomer, and carbopol.
